(19) United States
(12) Patent Application Publication
(10) Pub. No.: US 2024/0196744 A1
(43) Pub. Date: Jun. 13, 2024

(54) COMPOUND

(71) Applicant: Sumitomo Chemical Co., Ltd., Tokyo (JP)

(72) Inventors: Tania Zuberi, Godmanchester (GB); Michal Maciejczyk, Godmanchester (GB)

(73) Assignee: Sumitomo Chemical Co., Ltd., Tokyo (JP)

(21) Appl. No.: 18/099,870

(22) Filed: Jan. 20, 2023

(30) Foreign Application Priority Data
Nov. 28, 2022 (GB) 2217826.3

(51) Int. Cl. H10K 85/60; C07D 495/22; C07D 519/00

(52) U.S. Cl. CPC H10K 85/6576; C07D 495/22; C07D 519/00; H10K 85/626; H10K 85/655; H10K 30/30

(57) ABSTRACT

A compound formula (I):

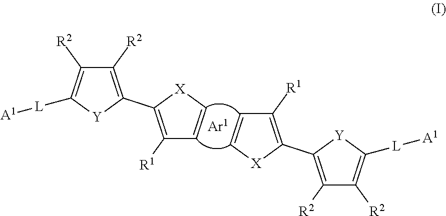

wherein: Ar¹ is a monocyclic or fused polycyclic group which, together with the rings it is fused to, forms a fused heteroaromatic group; Y is O, S or NR⁵⁵, wherein R⁵⁵ is H or a C₁₋₃₀ hydrocarbyl group; X is O or S; R¹ in each occurrence is independently H or a substituent with the proviso that at least one R¹ is an electron-withdrawing group; R² in each occurrence is independently H or a substituent; L is a direct bond or, together with R², forms an unsubstituted or substituted 5- or 6-membered aromatic or heteroaromatic ring; and A¹ in each occurrence is an electron-accepting group.

### RELATED APPLICATIONS

This application claims priority to United Kingdom Patent Application GB 2217826.3, filed Nov. 28, 2022, the contents of which are incorporated herein by reference.

### BACKGROUND

Embodiments of the present disclosure relate to electron-accepting compounds and more specifically compounds suitable for use as an electron-accepting material in a photoresponsive device.

An organic photodetector may contain a photoactive layer of a blend of an electron-donating material and an electron-accepting material between an anode and a cathode. Known electron-accepting materials include fullerenes and non-fullerene acceptors (NFAs).

Zhang Q et al, “The Curious Case of Fluorination of Conjugated Polymers for Solar Cells”, Acc Chem Res. 2017 Sep 19; 50(9): 2401-2409 describes fluorinated polymers for use in solar cells.

Park B et al, “Significant Dark Current Suppression in Organic Photodetectors Using Side Chain Fluorination of Conjugated Polymer”, Adv. Funct. Mater. 2022, 32, 2108026 describes fluorinated polymers for use in organic photodetectors.

Xin Y et al, “Enhancing thermal stability of nonfullerene organic solar cells via fluoro-side-chain engineering”, J. Mater. Chem. C, 2019,7, 9513-9522 describes fluorinated compounds for use in solar cells.

Chen Y et al, “Improving the performance of organic solar cells by side chain engineering of fused ring electron acceptors”, J. Mater. Chem. C, 2021, 9, 6937-6943 describes fluorinated compounds for use in solar cells.

Radford C et el, “Heteroatoms as Rotational Blocking Groups for Non-Fullerene Acceptors in Indoor Organic Solar Cells”, ACS Energy Lett. 2022, 7, 5, 1635-1641 describes compounds for use in solar cells.

Jiang S et al, “S⋅⋅⋅Cl intramolecular interaction: An efficient strategy to improve power conversion efficiency of organic solar cells”, Dyes and Pigments, volume 179, August 2020 describes compounds for use in solar cells.

Zuo et al, “PTB7-Th-Based Organic Photovoltaic Cells with a High V of over 1.0 V via Fluorination and Side Chain Engineering of Benzotriazole-Containing Nonfullerene Acceptors”, ACS Appl. Mater. Interfaces 2022, 14, 16, 18764-18772 describes compounds for use in photovoltaic cells.

Huang et al, “Modulating Chlorination Position on Polymer Donors for Highly Efficient Non-Fullerene Organic Solar Cells”, Solar RRL, Volume 5, Issue 10, October 2021 describes chlorinated polymers for use in solar cells.

Chai G et al, “Enhanced hindrance from phenyl outer side chains on nonfullerene acceptor enables unprecedented simultaneous enhancement in organic solar cell performances with 16.7% efficiency”, Nano Energy, Volume 76, October 2020 describes compounds for use in solar cells.

WO 2022/129137A1 describes compounds for use in organic photoresponsive devices.

WO 2020/008185A1 describes photoactive compounds and their use in organic electronic devices.

CN108659020B describes absorbing materials for use in solar cells.

### SUMMARY

The present disclosure provides a compound formula (I):

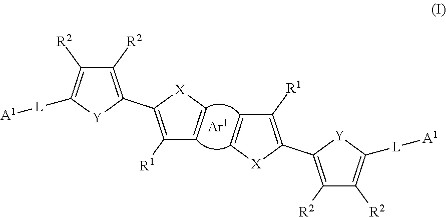

wherein: Ar¹ is a monocyclic or fused polycyclic group which, together with the rings it is fused to, forms a fused heteroaromatic group; Y is O, S or NR⁵⁵, wherein R⁵⁵ is H or a C₁₋₃₀ hydrocarbyl group; X is O or S; R¹ in each occurrence is independently H or a substituent with the proviso that at least one R¹ is an electron-withdrawing group; R² in each occurrence is independently H or a substituent; L is a direct bond or, together with R², forms an unsubstituted or substituted 5- or 6-membered aromatic or heteroaromatic ring; and A¹ in each occurrence is an electron-accepting group.

The present disclosure provides a composition comprising an electron-donating material and an electron-accepting material wherein the electron accepting material is a compound as described herein.

The present disclosure provides an organic electronic device comprising an active layer comprising a compound or composition as described herein.

Optionally, the organic electronic device is an organic photoresponsive device comprising a photoactive layer disposed between an anode and a cathode and wherein the photoactive layer comprises a compound as described herein. Optionally, the photoactive layer is a photoactive bulk heterojunction layer comprising a composition as described herein.

Optionally, the organic photoresponsive device is an organic photodetector.

The present disclosure provides a photosensor comprising a light source and an organic photodetector as described herein wherein the organic photodetector is configured to detect light emitted from the light source.

Optionally, the light source emits light having a peak wavelength of greater than 900 nm.

The present disclosure provides a formulation comprising a compound or composition as described herein dissolved or dispersed in one or more solvents.

The present disclosure provides a method of forming an organic electronic device as described herein wherein formation of the active layer comprises deposition of a formulation as described herein onto a surface and evaporation of the one or more solvents.

It has been found that the compounds described herein (compounds of formula (I) wherein at least one R¹ is an electron withdrawing group) have an increased thermal stability and stability in solution relative to similar compounds that do not include at least one electron withdrawing group R¹. Without wishing to be bound by theory, it is believed that the improvement in thermal stability is at least partially due to confirmation locking of the core heteroaromatic moiety with the furan, pyrrole or thiophene group(s) attached thereto. Such an improvement in thermal stability allows the compounds to be particularly useful in the manufacture of organic electronic devices, such as organic photoresponsive devices. Furthermore, it has been found that such compounds have/retain an optical absorption spectrum that is particularly useful in organic electronic devices, such as organic photoresponsive devices.

### DESCRIPTION OF DRAWINGS

The disclosed technology and accompanying figures describe some implementations of the disclosed technology.

FIG. 1 illustrates an organic photoresponsive device according to some embodiments.

The drawings are not drawn to scale and have various viewpoints and perspectives. The drawings are some implementations and examples. Additionally, some components and/or operations may be separated into different blocks or combined into a single block for the purposes of discussion of some of the embodiments of the disclosed technology. Moreover, while the technology is amenable to various modifications and alternative forms, specific embodiments have been shown by way of example in the drawings and are described in detail below. The intention, however, is not to limit the technology to the particular implementations described. On the contrary, the technology is intended to cover all modifications, equivalents, and alternatives falling within the scope of the technology as defined by the appended claims.

### DETAILED DESCRIPTION

Unless the context clearly requires otherwise, throughout the description and the claims, the words “comprise.” “comprising.” and the like are to be construed in an inclusive sense, as opposed to an exclusive or exhaustive sense; that is to say, in the sense of “including, but not limited to.” Additionally, the words “herein,” “above,” “below,” and words of similar import, when used in this application, refer to this application as a whole and not to any particular portions of this application. Where the context permits, words in the Detailed Description using the singular or plural number may also include the plural or singular number respectively. The word “or,” in reference to a list of two or more items, covers all of the following interpretations of the word: any of the items in the list, all of the items in the list, and any combination of the items in the list. References to a layer “over” another layer when used in this application means that the layers may be in direct contact or one or more intervening layers may be present. References to a layer “on” another layer when used in this application means that the layers are in direct contact. References to a specific atom include any isotope of that atom unless specifically stated otherwise.

The teachings of the technology provided herein can be applied to other systems, not necessarily the system described below. The elements and acts of the various examples described below can be combined to provide further implementations of the technology. Some alternative implementations of the technology may include not only additional elements to those implementations noted below, but also may include fewer elements.

These and other changes can be made to the technology in light of the following detailed description. While the description describes certain examples of the technology, and describes the best mode contemplated, no matter how detailed the description appears, the technology can be practiced in many ways. As noted above, particular terminology used when describing certain features or aspects of the technology should not be taken to imply that the terminology is being redefined herein to be restricted to any specific characteristics, features, or aspects of the technology with which that terminology is associated. In general, the terms used in the following claims should not be construed to limit the technology to the specific examples disclosed in the specification, unless the Detailed Description section explicitly defines such terms. Accordingly, the actual scope of the technology encompasses not only the disclosed examples, but also all equivalent ways of practicing or implementing the technology under the claims.

To reduce the number of claims, certain aspects of the technology are presented below in certain claim forms, but the applicant contemplates the various aspects of the technology in any number of claim forms.

In the following description, for the purposes of explanation, numerous specific details are set forth in order to provide a thorough understanding of implementations of the disclosed technology. It will be apparent, however, to one skilled in the art that embodiments of the disclosed technology may be practiced without some of these specific details. The present disclosure provides a compound of formula (I):

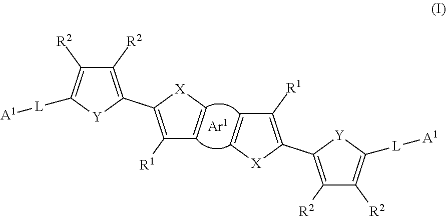

wherein: Ar¹ is a monocyclic or fused polycyclic group which, together with the rings it is fused to, forms a fused heteroaromatic group; Y in each occurrence is independently O, S or NR⁵⁵, wherein R⁵⁵ is H or a C₁₋₃₀ hydrocarbyl group; X in each occurrence is independently O or S; R¹ in each occurrence is independently H or a substituent with the proviso that at least one R¹ is an electron-withdrawing group; R² in each occurrence is independently H or a substituent; L is a direct bond or, together with R², forms an unsubstituted or substituted 5- or 6-membered aromatic or heteroaromatic ring; and A¹ in each occurrence is an electron-accepting group.

Each of the electron-accepting groups A¹ may have a lowest unoccupied molecular orbital (LUMO) level that is deeper (i.e., further from vacuum) than the LUMO of the electron-donating group represented by formula (II), preferably at least 1 eV deeper:

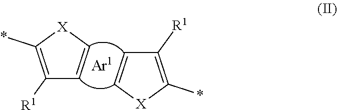

The LUMO levels of electron-accepting groups and electron-donating groups may be as determined by modelling the LUMO level of these groups, in which each bond to adjacent group is replaced with a bond to a hydrogen atom. Modelling may be performed using Gaussian09 software available from Gaussian using Gaussian09 with B3LYP (functional) and LACVP* (Basis set).

Ar¹ is a monocyclic or fused polycyclic group which, together with the rings it is fused to, forms a fused heteroaromatic group. In some embodiments, Ar¹ is a monocyclic or fused polycyclic group comprising at least one aromatic or heteroaromatic group. Optionally, Ar¹ may comprise one or more aromatic groups, such as phenyl; and/or one or more heteroaraomtic groups, such as thiophene or furan.

In some embodiments, the compound of formula (I) is represented by formula (Ia):

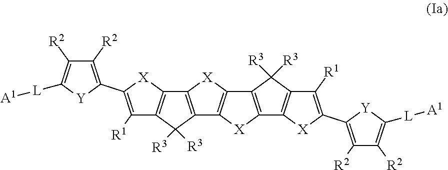

wherein: Ar¹, Y, X, R¹, R², L and A¹ are in each occurrence the same as defined in relation to formula (I); and R³ in each occurrence is independently H or a substituent, optionally wherein each R³ is a substituent.

In some embodiments, R¹ in each occurrence is independently H or a substituent selected from the group consisting of alkoxy, aldehydes, ketones, carboxylic acids, esters, halogens, and CN. Optionally, R¹ in each occurrence is independently H or a substituent selected from the group consisting of C₁₋₂₀ alkoxy, C₁₋₂₀ aldehydes, C₁₋₂₀ ketones, C₁₋₂₀ carboxylic acids, C₁₋₂₀ esters, halogens (such as F, Cl and Br), and CN. Optionally, R¹ in each occurrence is independently H or a substituent selected from the group consisting of C₁₋₁₂ alkoxy, C₁₋₁₂ aldehydes, C₁₋₁₂ ketones, C₁₋₁₂ carboxylic acids, C₁₋₁₂ esters, halogens (such as F. Cl and Br), and CN. For example, R¹ in each occurrence is independently H or a substituent selected from the group consisting of C₁₋₅ alkoxy, C₁₋₅ aldehydes, C₁₋₅ ketones, C₁₋₅ carboxylic acids, C₁₋₅ esters, halogens (such as F, Cl and Br), and CN.

Optionally, R¹ in each occurrence is independently H or a substituent selected from the group consisting of halogens and CN. For example, R¹ in each occurrence may be independently H or a substituent selected from the group consisting of F, Cl and Br.

Optionally, one R¹ is a substituent as defined herein. Optionally, two R¹s (i.e. each R¹) is a substituent as defined herein. In some embodiments, R¹ in each occurrence is independently a substituent as defined herein. When R¹ in each occurrence is a substituent, each R¹ may be the same or different. Optionally, each R¹ is different. Preferably, each R¹ is the same.

In some embodiments, R² is each occurrence is independently H or a substituent selected from the group consisting of F; CN; NO₂; C₁₋₂₀ alkyl wherein one or more non-adjacent C atoms may be replaced with O, S, NR⁶, COO or CO and one or more H atoms of the alkyl may be replaced with F; phenyl which is unsubstituted or substituted with one or more substituents; and —B(R¹⁴)₂; wherein R¹⁴ in each occurrence is a substituent, optionally a C₁₋₂₀ hydrocarbyl group; and R⁶ in each occurrence is independently selected from the group consisting of H; C₁₋₂₀ alkyl wherein one or more non-adjacent C atoms other than the C atom bound to N may be replaced with O, S, NR¹¹, COO or CO and one or more H atoms of the alkyl may be replaced with F; and phenyl which is unsubstituted or substituted with one or more substituents, optionally one or more C₁₋₁₂ alkyl groups wherein one or more non-adjacent C atoms of the alkyl may be replaced with O, S, NR¹¹, COO or CO and one or more H atoms of the alkyl may be replaced with F wherein R¹¹ is H or a C₁₋₂₀ hydrocarbyl group.

In some embodiments, R² in each occurrence is independently H or a substituent selected from the group consisting of F; CN; NO₂; C₁₋₂₀ alkyl wherein one or more non-adjacent C atoms may be replaced with O, S, NR⁶, COO or CO and one or more H atoms of the alkyl may be replaced with F; phenyl which is unsubstituted or substituted with one or more substituents; and —B(R¹⁴)₂; wherein R¹⁴ in each occurrence is a substituent, optionally a C₁₋₂₀ hydrocarbyl group; and R⁶ in each occurrence is independently selected from the group consisting of H; C₁₋₂₀ alkyl wherein one or more non-adjacent C atoms other than the C atom bound to N may be replaced with O, S, NR¹¹, COO or CO and one or more H atoms of the alkyl may be replaced with F; and phenyl which is unsubstituted or substituted with one or more substituents.

When one or more R¹⁴ is present and is a C₁₋₂₀ hydrocarbyl group; each R¹⁴ may independently be a C₁₋₁₂ hydrocarbyl group, a C₁₋₈ hydrocarbyl group, a C₁₋₅ hydrocarbyl group, or a C₁₋₃ hydrocarbyl group.

In some embodiments, R² groups may be linked to form a bicyclic ring, for example thienopyrazine, thienothiophene, thienopyrrole or thienothiazole.

R² in each occurrence may independently be H or a substituent selected from the group consisting of halogens, C₁₋₂₀ alkyl and C₁₋₁₉ alkoxy. Optionally, each R² is H, C₁₋₁₂ alkyl or C₁₋₁₂ alkoxy. Preferably, each R² is H, C₁₋₈ alkyl or C₁₋₈ alkoxy. For example, each R² is H, or C₁₋₃ alkoxy (such as methoxy or ethoxy).

In some embodiments, at least one R² is a substituent; optionally wherein at least two R²s are a substituent. Optionally, two, three or four R²s are a substituent. Preferably, two R²s are a substituent. More preferably, two R²s are a substituent and each substituent R² is directly attached to a different heterocyclic moiety. When more than one R² is a substituent, each substituent R² may be the same or different.

L is a direct bond or, together with R², forms an unsubstituted or substituted 5- or 6-membered aromatic or heteroaromatic ring. In some embodiments, L in each occurrence is independently a direct bond or, together with R², forms an unsubstituted or substituted 5- or 6-membered aromatic or heteroaromatic ring. Optionally, L in each occurrence is a direct bond. Optionally, L in each occurrence independently, together with R², forms an unsubstituted or substituted 5- or 6-membered aromatic or heteroaromatic ring. For example, L in each occurrence independently, together with R², forms a group selected from the group consisting of thienothiophene, thienopyrrole or thienothiazole. Preferably, when L together with R², forms an unsubstituted or substituted 5- or 6-membered aromatic or heteroaromatic ring, said ring is thienothiophene, thienopyrrole or thienothiazole, more preferably thienothiophene.

In some embodiments, when the compound is represented by formula (Ia), R³ in each occurrence is independently H or a substituent selected from the group consisting of C₁₋₂₀ alkyl wherein one or more non-adjacent C atoms may be replaced with O, S, NR⁶, COO or CO and one or more H atoms of the alkyl may be replaced with F; and phenyl which is unsubstituted or substituted with one or more substituents; and wherein R⁶ in each occurrence is independently selected from the group consisting of H; C₁₋₂₀ alkyl wherein one or more non-adjacent C atoms other than the C atom bound to N may be replaced with O, S, NR¹¹, COO or CO and one or more H atoms of the alkyl may be replaced with F; and phenyl which is unsubstituted or substituted with one or more substituents.

In some embodiments, when the compound is represented by formula (Ia), R³ in each occurrence is independently selected from the group consisting of C₁₋₂₀ alkyl wherein one or more non-adjacent C atoms may be replaced with O, S, NR⁶, COO or CO and one or more H atoms of the alkyl may be replaced with F; and phenyl which is unsubstituted or substituted with one or more substituents, optionally one or more C₁₋₁₂ alkyl groups wherein one or more non-adjacent C atoms may be replaced with O, S, NR⁶, COO or CO and one or more H atoms of the alkyl may be replaced with F; and wherein R⁶ in each occurrence is independently selected from H; C₁₋₂₀ alkyl wherein one or more non-adjacent C atoms other than the C atom bound to N may be replaced with O, S, NR¹¹, COO or CO and one or more H atoms of the alkyl may be replaced with F; and phenyl which is unsubstituted or substituted with one or more substituents, optionally one or more C₁₋₁₂ alkyl groups wherein one or more non-adjacent C atoms of the alkyl may be replaced with O, S, NR¹¹, COO or CO and one or more H atoms of the alkyl may be replaced with F wherein R¹¹ is H or a C₁₋₂₀ hydrocarbyl group.

Optionally, when the compound is represented by formula (Ia), R³ in each occurrence is independently selected from the group consisting of C₁₋₁₂ alkyl wherein one or more non-adjacent C atoms may be replaced with O, S, COO or CO and one or more H atoms of the alkyl may be replaced with F; and phenyl which is unsubstituted or substituted with one or more substituents, optionally one or more C₁₋₁₂ alkyl groups wherein one or more non-adjacent C atoms may be replaced with O, S, COO or CO and one or more H atoms of the alkyl may be replaced with F.

Optionally, when the compound is represented by formula (Ia), R³ in each occurrence is independently selected from the group consisting of C₁₋₈ alkyl wherein one or more non-adjacent C atoms may be replaced with O, S, COO or CO and one or more H atoms of the alkyl may be replaced with F; and phenyl which is unsubstituted or substituted with one or more substituents, optionally one or more C₁₋₈ alkyl groups wherein one or more non-adjacent C atoms may be replaced with O, S, COO or CO and one or more H atoms of the alkyl may be replaced with F.

Optionally, when the compound is represented by formula (Ia), R³ in each occurrence is independently selected from the group consisting of C₁₋₅ alkyl wherein one or more non-adjacent C atoms may be replaced with O, S, COO or CO and one or more H atoms of the alkyl may be replaced with F; and phenyl which is substituted with one or more substituents selected from the group consisting of C₁₋₅ alkyl groups wherein one or more non-adjacent C atoms may be replaced with O, S, COO or CO and one or more H atoms of the alkyl may be replaced with F.

In some embodiments, when the compound is represented by formula (Ia), at least one R³ is a substituent; optionally wherein at least two or three R³s are a substituent. Optionally, two, three or four R³s are a substituent. Preferably, four R³s (i.e. each R³) are independently a substituent as defined herein. When more than one R³ is a substituent, each substituent R³ may be the same or different. Preferably, each R³ is the same.

In some embodiments, Y in each occurrence is independently O or S; optionally S. For example, each Y is S.

In some embodiments, X in each occurrence is independently O or S; optionally S. For example, each X is S.

Unless otherwise specified, any substituent described herein may be independently selected from the group consisting of C₁₋₁₂ alkyl wherein one or more non-adjacent C atoms may be replaced with O, S, NR⁶, COO or CO and one or more H atoms of the alkyl may be replaced with F; and an aromatic group, optionally phenyl, which is unsubstituted or substituted with one or more substituents selected from F and C₁₋₁₂ alkyl wherein one or more non-adjacent C atoms may be replaced with O, S, NR⁶, COO or CO; and wherein NR⁶ is as described herein.

The present disclosure provides a compound of formula (I):

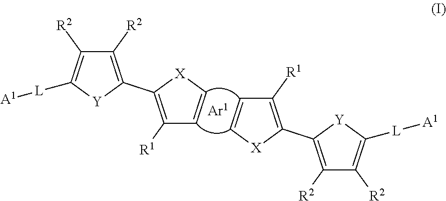

wherein: Ar¹ is a monocyclic or fused polycyclic group which, together with the rings it is fused to, forms a fused heteroaromatic group; Y in each occurrence is independently O, S or NR⁵⁵, wherein R⁵⁵ is H or a C₁₋₃₀ hydrocarbyl group; X in each occurrence is independently O or S; R¹ in each occurrence is independently H or a substituent; R² in each occurrence is independently H or a substituent with the proviso that at least one R² is an electron-withdrawing group; L is a direct bond or, together with R², forms an unsubstituted or substituted 5- or 6-membered aromatic or heteroaromatic ring; and A¹ in each occurrence is an electron-accepting group.

In some embodiments, the R² group closest to X is an electron-withdrawing group. In some embodiments, the R² group furthest from X is an electron-withdrawing group. In some embodiments, each R² is an electron-withdrawing group.

### Electron-Accepting Groups A¹

The monovalent acceptor groups A¹ may each independently be selected from any such units known to the skilled person. A¹ may be the same or different, preferably the same.

Exemplary monovalent acceptor groups include, without limitation, groups of formulae (IXa)-(IXq)

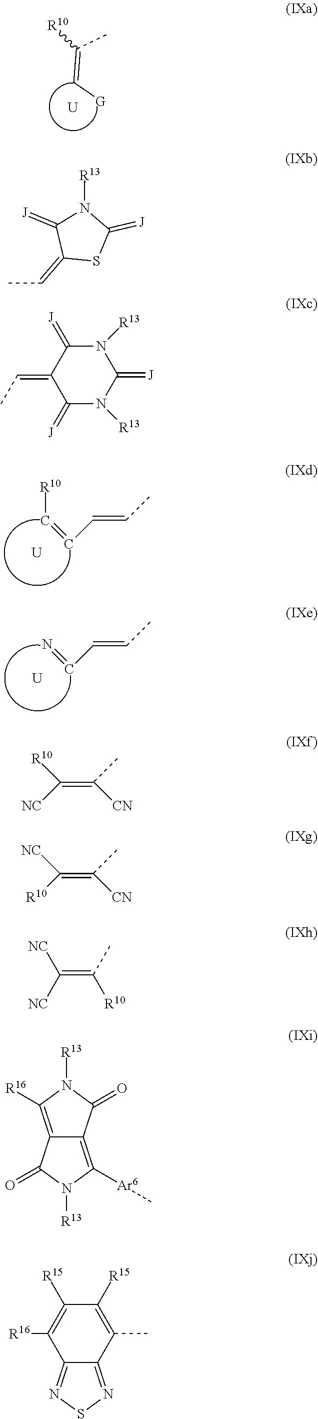

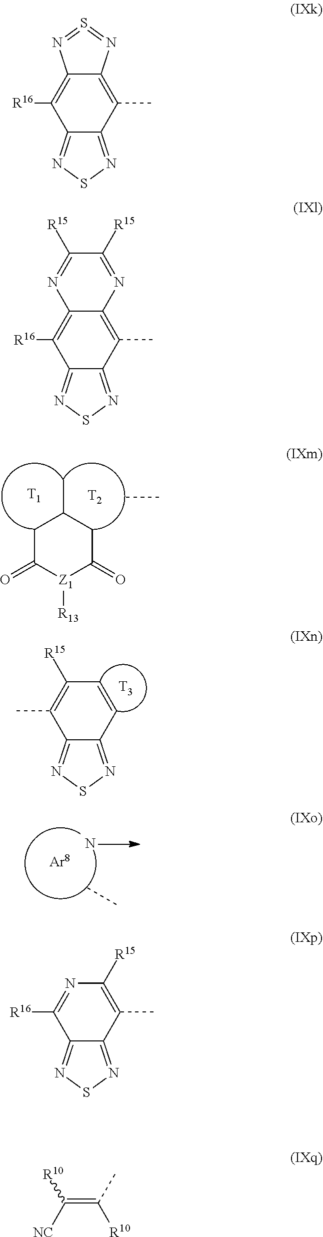

U is a 5- or 6-membered ring which is unsubstituted or substituted with one or more substituents and which may be fused to one or more further rings. Optionally, each substituent may be independently selected from the group consisting of C₁₋₁₂ alkyl wherein one or more non-adjacent C atoms may be replaced with O, S, NR⁶, COO or CO and one or more H atoms of the alkyl may be replaced with F; and an aromatic group, optionally phenyl, which is unsubstituted or substituted with one or more substituents selected from F and C₁₋₁₂ alkyl wherein one or more non-adjacent C atoms may be replaced with O, S, NR⁶, COO or CO; and wherein NR⁶ is as described herein.

G is C—O, C═S SO, SO2, NR³³ or C(R³³)₂ wherein R³³ is CN or COOR⁴⁰ and R⁴⁰ in each occurrence is independently H or a substituent, preferably H or a C₁₋₂₀ hydrocarbyl group. G is preferably C═O or SO2, more preferably C═O.

Optionally, each R⁶ of any NR⁶ or PR⁶ described anywhere herein is independently selected from H; C₁₋₂₀ alkyl wherein one or more non-adjacent C atoms other than the C atom bound to N or P may be replaced with O, S, NR¹¹, COO or CO and one or more H atoms of the alkyl may be replaced with F; and phenyl which is unsubstituted or substituted with one or more substituents, optionally one or more C₁₋₁₂ alkyl groups wherein one or more non-adjacent C atoms of the alkyl may be replaced with O, S, NR¹¹, COO or CO and one or more H atoms of the alkyl may be replaced with F wherein R¹¹ is H or a C₁₋₂₀ hydrocarbyl group.

A C₁₋₂₀ hydrocarbyl group as described anywhere is preferably selected from C₁₋₂₀ alkyl; unsubstituted phenyl; and phenyl substituted with one or more C₁₋₁₂ alkyl groups

The N atom of formula (IXe) may be unsubstituted or substituted.

R¹⁰ is H or a substituent, preferably a substituent selected from the group consisting of C₁₋₁₂ alkyl wherein one or more non-adjacent C atoms may be replaced with O, S, NR⁶, COO or CO and one or more H atoms of the alkyl may be replaced with F; and an aromatic group, optionally phenyl, which is unsubstituted or substituted with one or more substituents selected from F and C₁₋₁₂ alkyl wherein one or more non-adjacent C atoms may be replaced with O, S, NR⁶, COO or CO.

Preferably, R¹⁰ is H.

J is O or S, preferably O.

R¹³ in each occurrence is a substituent, optionally C₁₋₁₂ alkyl wherein one or more non-adjacent C atoms may be replaced with O, S, NR⁶, COO or CO and one or more H atoms of the alkyl may be replaced with F.

R¹⁵ in each occurrence is independently H; F; C₁₋₁₂ alkyl wherein one or more non-adjacent C atoms may be replaced with O, S, NR⁶, COO or CO and one or more H atoms of the alkyl may be replaced with F; aromatic group Ar², optionally phenyl, which is unsubstituted or substituted with one or more substituents selected from F and C₁₋₁₂ alkyl wherein one or more non-adjacent C atoms may be replaced with O, S, NR⁶, COO or CO; or a group selected from:

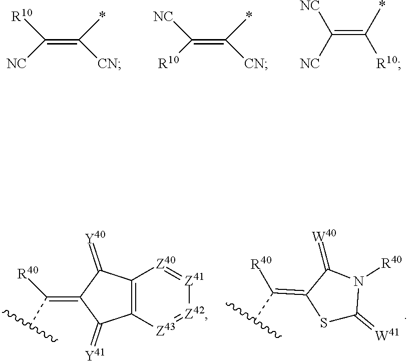

R¹⁶ is H or a substituent, preferably a substituent selected from:

—(Ar³)_{w} wherein Ar³ in each occurrence is independently an unsubstituted or substituted aryl or heteroaryl group, preferably thiophene, and w is 1, 2 or 3;

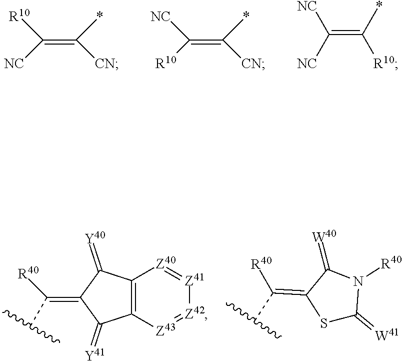

and

C₁₋₁₂ alkyl wherein one or more non-adjacent C atoms may be replaced with O, S, NR⁶, COO or CO and one or more H atoms of the alkyl may be replaced with F.

Ar⁶ is a 5-membered heteroaromatic group, preferably thiophene or furan, which is unsubstituted or substituted with one or more substituents.

Substituents of Ar³ and Ar⁶, where present, are optionally selected from C₁₋₁₂ alkyl wherein one or more non-adjacent C atoms may be replaced with O, S, NR⁶, COO or CO and one or more H atoms of the alkyl may be replaced with F.

T¹, T² and T³ each independently represent an aryl or a heteroaryl ring, optionally benzene, which may be fused to one or more further rings. Substituents of T¹, T² and T³, where present, are optionally selected from non-H groups of R²⁵. In a preferred embodiment, T³ is benzothiadiazole.

z¹ is N or P.

Ar⁸ is a fused heteroaromatic group which is unsubstituted or substituted with one or more substituents, optionally one or more non-H substituents R¹⁰, and which is bound to an aromatic C atom of formula (I) or formula (la) and to a boron containing substituent R² of formula (I) or formula (Ia).

Preferred groups A¹ are groups having a non-aromatic carbon-carbon bond which is bound directly to formula (I) or formula (Ia).

Preferably at least one A¹, preferably both groups A¹, are a group of formula (IXa-1):

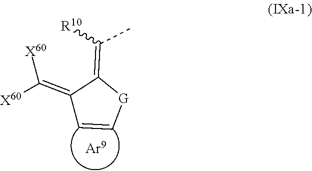

wherein:
G is as described above and is preferably C═O or SO₂, more preferably C═O;
R¹⁰ is as described above;
Ar⁹ is an unsubstituted or substituted monocyclic or fused aromatic or heteroaromatic group, preferably benzene or a monocyclic or bicyclic heteroaromatic group having C or N ring atoms only; and
X⁶⁰ are each independently CN, CF₃ or COOR⁴⁰ wherein R⁴⁰ in each occurrence is H or a substituent, preferably H or a C₁₋₂₀ hydrocarbyl group. Preferably, each X⁶⁰ is CN.
Ar⁹ may be unsubstituted or substituted with one or more substituents. Substituents of Ar⁹ are preferably selected from groups R¹² as described below.

Optionally, the group of formula (IXa-1) has formula (IXa-2):

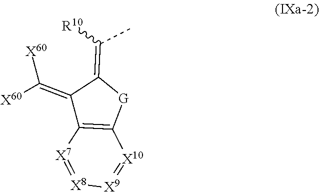

wherein each X⁷-X¹⁰ is independently CR¹² or N wherein R¹² in each occurrence is H or a substituent selected from C₁₋₂₀ hydrocarbyl and an electron withdrawing group. Preferably, the electron withdrawing group is F, Cl, Br or CN, more preferably F, Cl or CN; and for example For CN.

The C₁₋₂₀ hydrocarbyl group R¹² may be selected from C₁₋₂₀ alkyl; unsubstituted phenyl; and phenyl substituted with one or more C₁₋₁₂ alkyl groups.

In a particularly preferred embodiment, each of X⁷-X¹⁰ is CR¹² and each R¹² is independently selected from H or an electron-withdrawing group, preferably H, F or CN. According to this embodiment, R¹² of X⁸ and X⁹ is an electron-withdrawing group, preferably F or CN.

Exemplary groups of formula (IXd) include:

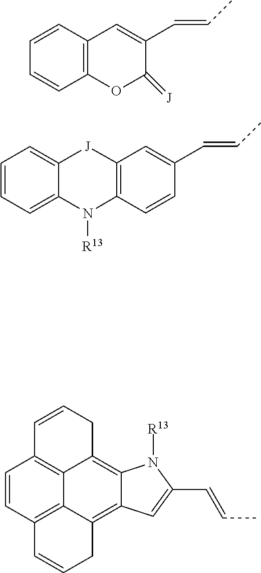

Exemplary groups of formula (IXe) include:

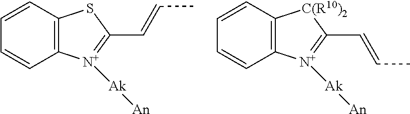

An exemplary group of formula (IXq) is:

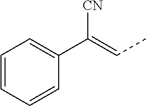

An exemplary group of formula (IXg) is:

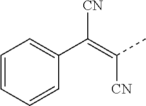

An exemplary group of formula (IXj) is:

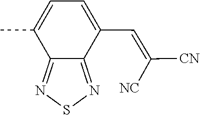

wherein Ak is a C₁₋₁₂ alkylene chain in which one or more C atoms may be replaced with O, S, NR⁶, CO or COO; An is an anion, optionally —SO₃⁻; and each benzene ring is independently unsubstituted or substituted with one or more substituents selected from substituents described with reference to R¹⁰.

Exemplary groups of formula (IXm) are:

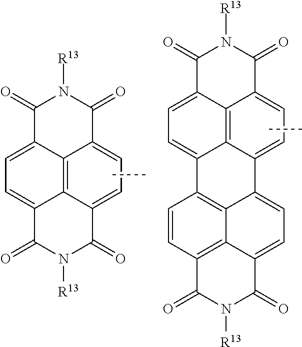

An exemplary group of formula (IXn) is:

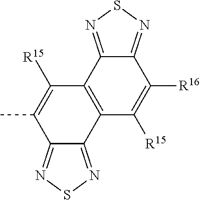

Groups of formula (IXo) are bound directly to formula (I) or formula (Ia) substituted with a R² group of formula —B(R¹⁴)₂ wherein R¹⁴ in each occurrence is a substituent, optionally a C₁₋₂₀ hydrocarbyl group; → is a bond to the boron atom —B(R¹⁴)₂; and --- is a C—C bond between formula (IXo) and formula (I) or formula (Ia). For example, → is a bond to the boron atom —B(R¹⁴)₂ of the R² group which is spatially closest to a nitrogen atom of the group of formula (IXo).

Optionally, R¹⁴ is selected from C₁₋₁₂ alkyl; unsubstituted phenyl; and phenyl substituted with one or more C₁₋₁₂ alkyl groups.

The group of formula (IXo), formula (I) and the B(R¹⁴)₂ substituent R² of formula (I) may be linked together to form a 5- or 6-membered ring.

Optionally groups of formula (IXo) are selected from:

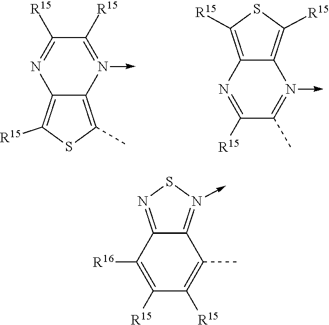

If a C atom of an alkyl group as described anywhere herein is replaced with another atom or group, the replaced C atom may be a terminal C atom of the alkyl group or a non-terminal C-atom.

By “non-terminal C atom” of an alkyl group as used anywhere herein means a C atom other than the C atom of the methyl group at the end of an n-alkyl chain or the C atoms of the methyl groups at the ends of a branched alkyl chain.

If a terminal C atom of a group as described anywhere herein is replaced then the resulting group may be an anionic group comprising a countercation, e.g., an ammonium or metal countercation, preferably an ammonium or alkali metal cation.

A C atom of an alkyl substituent group which is replaced with another atom or group as described anywhere herein is preferably a non-terminal C atom, and the resultant substituent group is preferably non-ionic.

### Bridging Units

Bridging units in formula (I) are represented by formula (III):

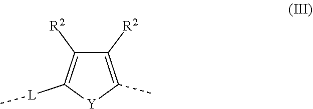

Optionally, the bridging unit in formula (I) may be selected from units of formulae (VIa)-(VIo):

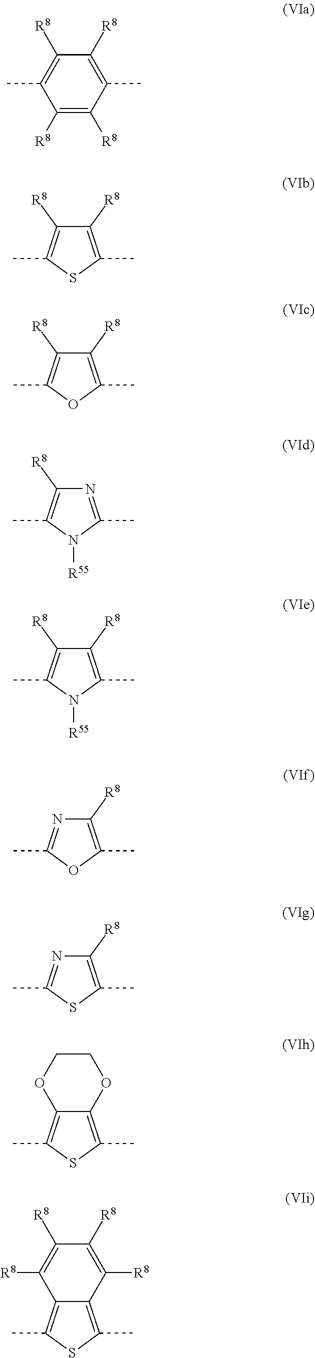

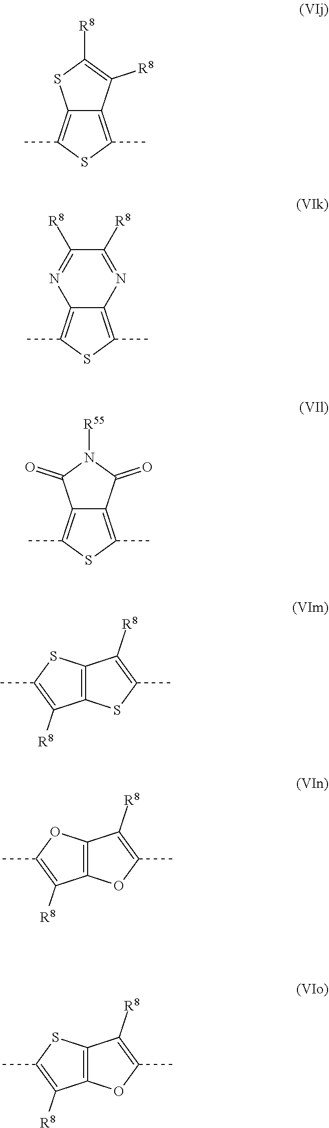

wherein R⁵⁵ is H or a substituent, optionally H or a C₁₋₂₀ hydrocarbyl group; and R⁸ in each occurrence is independently H or a substituent, preferably H or a substituent selected from F; CN; NO₂; C₁₋₂₀ alkyl wherein one or more non-adjacent C atoms may be replaced with O, S, NR⁶, COO or CO and one or more H atoms of the alkyl may be replaced with F; phenyl which is unsubstituted or substituted with one or more substituents; and —B(R¹⁴)₂ wherein R¹⁴ in each occurrence is a substituent, optionally a C₁₋₂₀ hydrocarbyl group. R⁸ groups of formulae (VIa), (VIb) and (VIc) may be linked to form a bicyclic ring, for example thienopyrazine.

R⁸ is preferably H, C₁₋₂₀ alkyl or C₁₋₁₉ alkoxy.

R⁸ groups of formulae (VIa), (VIb) and (VIc) may be linked to form an optionally substituted bicyclic ring.

### Electron-Donating Groups

The compound of formula (I) comprises an electron-donating group represented by formula (II):

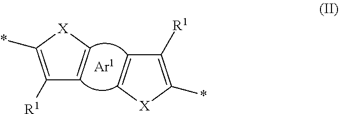

Exemplary electron-donating groups of formula (II) of the compound of formula (I) include groups of formulae (VIIa)-(VIIi):

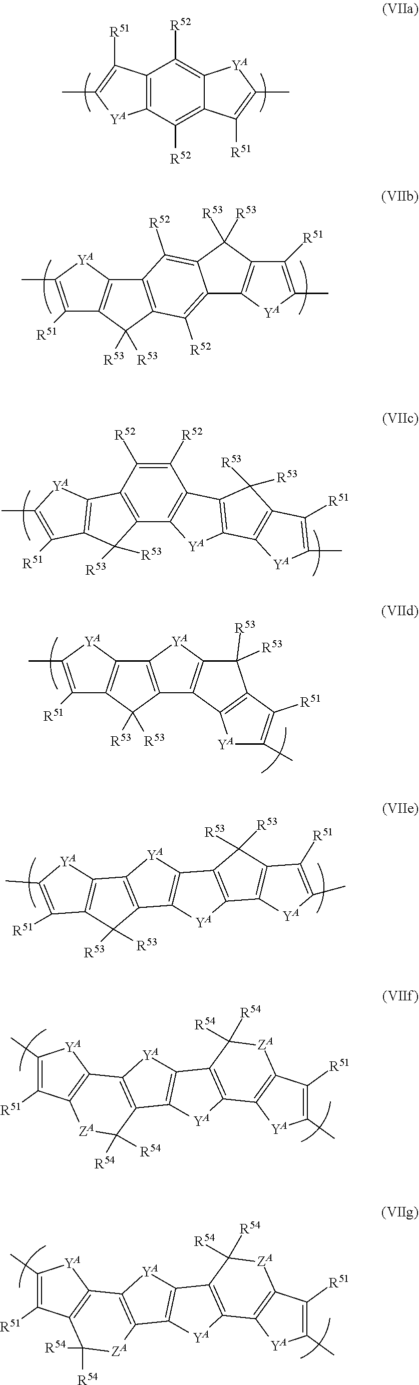

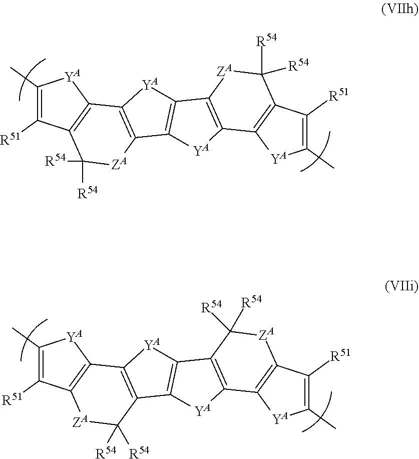

wherein Y^{A} in each occurrence is independently O, S or NR⁵⁵, Y^{A1} in each occurrence is independently O or S; X^{A} is C or Si; Z^{A} in each occurrence is O, CO, S, NR⁵⁵ or C(R⁵⁴)₂; R⁵² R⁵⁴ and R⁵⁵ independently in each occurrence is H or a substituent; R⁵³ independently in each occurrence is a substituent; Ar⁴ is an optionally substituted monocyclic or fused heteroaromatic group; and R⁵¹ is the same as described for R¹ in relation to formula (I) and/or (Ia).

Optionally, R⁵¹ and R⁵² independently in each occurrence are selected from H; F; C₁₋₂₀ alkyl wherein one or more non-adjacent C atoms may be replaced with O, S, NR⁶, COO or CO and one or more H atoms of the alkyl may be replaced with F; and an aromatic or heteroaromatic group Ar³ which is unsubstituted or substituted with one or more substituents.

In some embodiments, Ar³ may be an aromatic group, e.g., phenyl.

Ar⁴ is preferably selected from optionally substituted oxadiazole, thiadiazole, triazole, and 1,4-diazine. In the case where Art is 1,4-diazine, the 1,4-diazine may be fused to a further heterocyclic group, optionally a group selected from optionally substituted oxadiazole, thiadiazole, triazole, 1,4-diazine and succinimide.

The one or more substituents of Ar³, if present, may be selected from C₁₋₁₂ alkyl wherein one or more non-adjacent C atoms may be replaced with O, S, NR⁶, COO or CO and one or more H atoms of the alkyl may be replaced with F.

Preferably, each R⁵⁴ is selected from the group consisting of:
H;
F;
linear, branched or cyclic C₁₋₂₀ alkyl wherein one or more non-adjacent C atoms may be replaced by O, S, NR¹⁷, CO or COO wherein R¹⁷ is a C₁₋₁₂ hydrocarbyl and one or more H atoms of the C₁₋₂₀ alkyl may be replaced with F; and
a group of formula (Ak)u-(Ar⁷)v wherein Ak is a C₁₋₂₀ alkylene chain in which one or more non-adjacent C atoms may be replaced with O, S, NR⁶, CO or COO; u is 0 or 1; Ar⁷ in each occurrence is independently an aromatic or heteroaromatic group which is unsubstituted or substituted with one or more substituents; and v is at least 1, optionally 1, 2 or 3.

Substituents of Ar⁷, if present, are preferably selected from F; Cl; NO₂; CN; and C₁₋₂₀ alkyl wherein one or more non-adjacent C atoms may be replaced with O, S, NR⁶, CO or COO and one or more H atoms may be replaced with F. Preferably, Ar⁷ is phenyl.

Optionally, R⁵³ independently in each occurrence is selected from C₁₋₂₀ alkyl wherein one or more non-adjacent C atoms may be replaced with O, S, NR⁶, COO or CO and one or more H atoms of the alkyl may be replaced with F; and phenyl which is unsubstituted or substituted with one or more substituents, optionally one or more C₁₋₁₂ alkyl groups wherein one or more non-adjacent C atoms may be replaced with O, S, NR⁶, COO or CO and one or more H atoms of the alkyl may be replaced with F.

Preferably, R⁵⁵ as described anywhere herein is H or C₁₋₃₀ hydrocarbyl group; optionally a C₁₋₂₀ hydrocarbyl group or optionally a C₁₋₁₂ hydrocarbyl group.

In a preferred embodiment, the electron-donating group of formula (II) of the compound of formula (I) is a group of formula (VIIe).

In some embodiments, the electron-donating group of formula (II) of the compound of formula (I) comprises two or more groups of formulae (VIIa)-(VIIi) bound together (e.g. in a chain).

In these embodiments, the chain of electron-donating groups of formula (II), respectively, may be linked in any orientation.

Exemplary compounds of formula (I) include, without limitation:

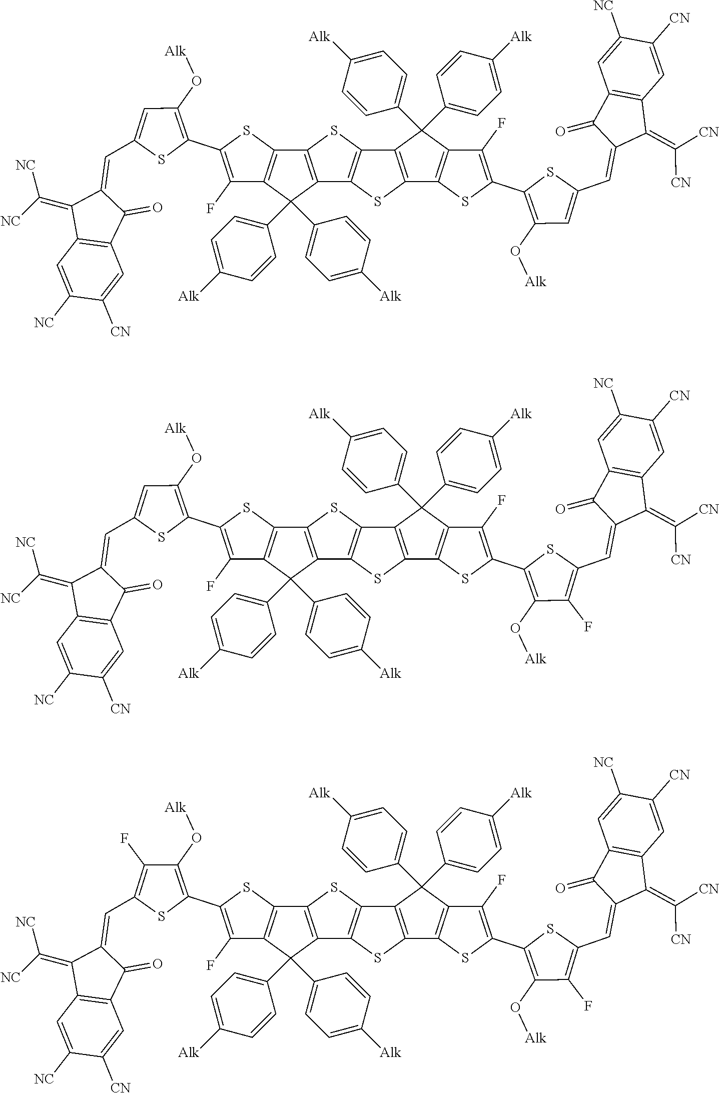

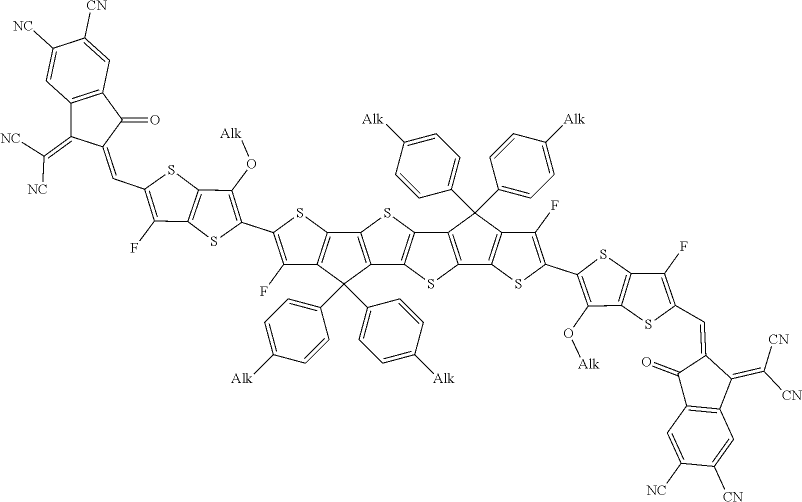

wherein Alk in each occurrence is independently a C₁₋₁₂ alkyl group.

### Electron-Donating Material

A photoactive layer as described herein comprises an electron-donating material and a compound of formula (I) as described herein, either in the same layer (a bulk heterojunction layer) or separate sub-layers.

Exemplary donor materials are disclosed in, for example, WO2013/051676, the contents of which are incorporated herein by reference.

The electron-donating material may be a non-polymeric or polymeric material.

In a preferred embodiment the electron-donating material is an organic conjugated polymer, which can be a homopolymer or copolymer including alternating, random or block copolymers. The conjugated polymer is preferably a donor-acceptor polymer comprising alternating electron-donating repeat units and electron-accepting repeat units.

Preferred are non-crystalline or semi- crystalline conjugated organic polymers.

Further preferably the electron-donating polymer is a conjugated organic polymer with a low bandgap, typically between 2.5 eV and 1.5 eV, preferably between 2.3 eV and 1.8 eV.

Optionally, the electron-donating polymer has a HOMO level no more than 5.5 eV from vacuum level. Optionally, the electron-donating polymer has a HOMO level at least 4.1 eV from vacuum level. As exemplary electron-donating polymers, polymers selected from conjugated hydrocarbon or heterocyclic polymers including polyacene, polyaniline, polyazulene, polybenzofuran, polyfluorene, polyfuran, polyindenofluorene, polyindole, polyphenylene, polypyrazoline, polypyrene, polypyridazine, polypyridine, polytriarylamine, poly(phenylene vinylene), poly(3-substituted thiophene), poly(3,4-bisubstituted thiophene), polyselenophene, poly(3-substituted selenophene), poly(3,4-bisubstituted selenophene), poly(bisthiophene), poly(terthiophene), poly(bisselenophene), poly(terselenophene), polythieno[2,3-b]thiophene, polythieno[3,2-b]thiophene, polybenzothiophene, polybenzo[1,2-b:4,5-b′]dithiophene, polyisothianaphthene, poly(monosubstituted pyrrole), poly(3,4-bisubstituted pyrrole), poly-1,3,4-oxadiazoles, polyisothianaphthene, derivatives and co-polymers thereof may be mentioned.

Preferred examples of donor polymers are copolymers of polyfluorenes and polythiophenes, each of which may be substituted, and polymers comprising benzothiadiazole-based and thiophene-based repeating units, each of which may be substituted.

A particularly preferred donor polymer comprises a repeat unit of formula (X):

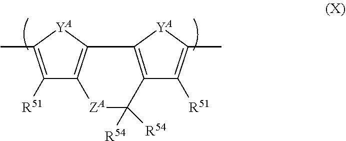

wherein Y^{A}, Z^{A}, R⁵¹ and R⁵⁴ are as described above.

Another particularly preferred donor polymer comprises repeat units of formula (XI):

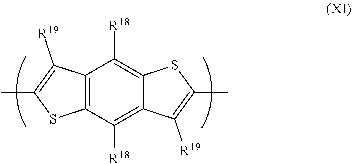

wherein R¹⁸ and R¹⁹ are each independently selected from H; F; C₁₋₁₂ alkyl wherein one or more non-adjacent, non-terminal C atoms may be replaced with O, S, COO or CO and one or more H atoms of the alkyl may be replaced with F; or an aromatic or heteroaromatic group Ar⁶ which is unsubstituted or substituted with one or more substituents selected from F and C₁₋₁₂ alkyl wherein one or more non-adjacent, non-terminal C atoms may be replaced with O, S, COO or CO.

The donor polymer is preferably a donor-acceptor (DA) copolymer comprising a donor repeat unit, for example a repeat unit of formula (X) or (XI), and an acceptor repeat unit, for example divalent electron-accepting units A¹ as described herein provided as polymeric repeat units.

### Organic Electronic Device

A compound of formula (I) may be provided as an active layer of an organic electronic device. In a preferred embodiment, a photoactive layer of an organic photoresponsive device, more preferably an organic photodetector, comprises a compound of formula (I) as described herein.

In some embodiments, the photoactive layer comprises an electron-accepting sub-layer and an electron-donating sub-layer directly adjacent to and in contact with the electron-accepting sub-layer.

In some embodiments, the photoactive layer is a photoactive bulk heterojunction layer comprising or consisting of an electron-donating material and an electron-accepting compound of formula (I) as described herein.

In some embodiments, the photoactive layer contains two or more accepting materials and/or two or more electron-accepting materials.

In the case of a bulk heterojunction layer, the weight of the electron-donating material(s) to the electron-accepting material(s) is optionally from about 1:0.5 to about 1:2, preferably about 1:1.1 to about 1:2.

Preferably, the electron-donating material has a type II interface with the electron-accepting material, i.e. the electron-donating material has a shallower HOMO and LUMO that the corresponding HOMO and LUMO levels of the electron-accepting material. Preferably, the compound of formula (I) has a HOMO level that is at least 0.05 eV deeper, optionally at least 0.10 eV deeper, than the HOMO of the electron-donating material.

Optionally, the gap between the HOMO level of the electron-donating material and the LUMO level of the electron-accepting compound of formula (I) or (II) is less than 1.4 eV.

Unless stated otherwise, HOMO and LUMO levels of materials as described herein are as measured by square wave voltammetry (SWV).

In SWV, the current at a working electrode is measured while the potential between the working electrode and a reference electrode is swept linearly in time. The difference current between a forward and reverse pulse is plotted as a function of potential to yield a voltammogram. Measurement may be with a CHI 660D Potentiostat.

The apparatus to measure HOMO or LUMO energy levels by SWV may comprise a cell containing 0.1 M tertiary butyl ammonium hexafluorophosphate in acetonitrile; a 3 mm diameter glassy carbon working electrode; a platinum counter electrode and a leak free Ag/AgCl reference electrode.

Ferrocene is added directly to the existing cell at the end of the experiment for calculation purposes where the potentials are determined for the oxidation and reduction of ferrocene versus Ag/AgCl using cyclic voltammetry (CV).

The sample is dissolved in toluene (3 mg/ml) and spun at 3000 rpm directly on to the glassy carbon working electrode.

LUMO=4.8-E ferrocene (peak to peak average)−E reduction of sample (peak maximum).

HOMO=4.8-E ferrocene (peak to peak average)+E oxidation of sample (peak maximum).

A typical SWV experiment runs at 15 Hz frequency; 25 mV amplitude and 0.004 V increment steps. Results are calculated from 3 freshly spun film samples for both the HOMO and LUMO data.

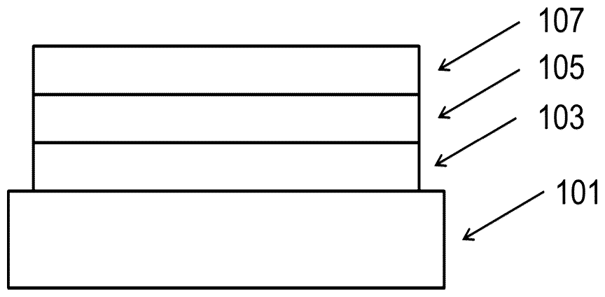

FIG. 1 illustrates an organic photoresponsive device according to some embodiments of the present disclosure. The organic photoresponsive device comprises a cathode 103, an anode 107 and a photoactive bulk heterojunction layer 105 disposed between the anode and the cathode. The organic photoresponsive device may be supported on a substrate 101, optionally a glass or plastic substrate.

Each of the anode and cathode may independently be a single conductive layer or may comprise a plurality of layers.

At least one of the anode and cathode is transparent so that light incident on the device may reach the bulk heterojunction layer. In some embodiments, both of the anode and cathode are transparent. The transmittance of a transparent electrode may be selected according to an emission wavelength of a light source for use with the organic photodetector.

FIG. 1 illustrates an arrangement in which the cathode is disposed between the substrate and the anode. In other embodiments, the anode may be disposed between the cathode and the substrate.

FIG. 1 illustrates an arrangement in which the photoactive layer is a bulk heterojunction layer. In other embodiments, the photoactive layer comprises an electron-accepting sub-layer and an electron-donating sub-layer directly adjacent to and in contact with the electron-accepting sub-layer.

The organic photoresponsive device may comprise layers other than the anode, cathode and photoactive layer shown in FIG. 1. In some embodiments, a hole-transporting layer is disposed between the anode and the photoactive layer. In some embodiments, an electron-transporting layer is disposed between the cathode and the photoactive layer. In some embodiments, a work function modification layer is disposed between the photoactive layer and the anode, and/or between the photoactive layer and the cathode.

The area of the OPD may be less than about 3 cm², less than about 2 cm², less than about 1 cm², less than about 0.75 cm², less than about 0.5 cm² or less than about 0.25 cm². Optionally, each OPD may be part of an OPD array wherein each OPD is a pixel of the array having an area as described herein, optionally an area of less than 1 mm2, optionally in the range of 0.5 micron²-900 micron².

The substrate may be, without limitation, a glass or plastic substrate. The substrate can be an inorganic semiconductor. In some embodiments, the substrate may be silicon. For example, the substrate can be a wafer of silicon. The substrate is transparent if, in use, incident light is to be transmitted through the substrate and the electrode supported by the substrate.

A bulk heterojunction layer as described herein contains an electron-accepting compound of formula (I) as described herein and an electron-donating compound. The bulk heterojunction layer may consist of these materials or may comprise one or more further materials, for example one or more further electron-donating materials and/or one or more further electron-accepting compounds.

An electron-accepting sub-layer as described herein may consist of a single electron-accepting compound of formula (I) or may comprise one or more further materials, for example one or more further electron-accepting materials.

An electron-donating sub-layer as described herein may consist of a single electron-donating material or may comprise one or more further materials, for example one or more further electron-donating materials.

### Fullerene

In some embodiments, a bulk heterojuction layer or electron-accepting sub-layer contains a compound of formula (I) and one or more further electron-accepting materials. Preferred further electron-accepting materials are fullerenes. The compound of formula (I):fullerene acceptor weight ratio may be in the range of about 1:0.1-1:1, preferably in the range of about 1:0.1-1:0.5.

Fullerenes may be selected from, without limitation, C₆₀, C₇₀, C₇₆, C₇₈ and C₈₄ fullerenes or a derivative thereof, including, without limitation, PCBM-type fullerene derivatives including phenyl-C₆₁-butyric acid methyl ester (C₆₀PCBM), TCBM-type fullerene derivatives (e.g. tolyl-C₆₁-butyric acid methyl ester (C₆₀TCBM)), and ThCBM-type fullerene derivatives (e.g. thienyl-C₆₁-butyric acid methyl ester (C₆₀ThCBM).

Fullerene derivatives may have formula (V):

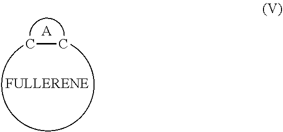

wherein A, together with the C—C group of the fullerene, forms a monocyclic or fused ring group which may be unsubstituted or substituted with one or more substituents.

Exemplary fullerene derivatives include formulae (Va), (Vb) and (Vc):

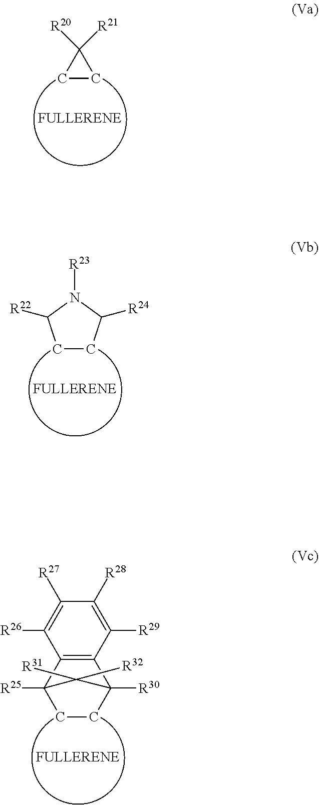

wherein R²⁰-R³² are each independently H or a substituent.

Substituents R²⁰-R³² are optionally and independently in each occurrence selected from the group consisting of aryl or heteroaryl, optionally phenyl, which may be unsubstituted or substituted with one or more substituents; and C₁₋₂₀ alkyl wherein one or more non-adjacent C atoms may be replaced with O, S, NR⁶, CO or COO and one or more H atoms may be replaced with F.

Substituents of aryl or heteroaryl, where present, are optionally selected from C₁₋₁₂ alkyl wherein one or more non-adjacent C atoms may be replaced with O, S, NR⁶, CO or COO and one or more H atoms may be replaced with F.

### Formulations

Formation of an electron-accepting sub-layer of a photoactive layer or a photoactive bulk heterojunction layer as described herein preferably comprises deposition of a formulation comprising electron-accepting material(s) including the compound of formula (I) and any other components of the electron-accepting sub-layer or bulk heterojunction layer dissolved or dispersed in a solvent or a mixture of two or more solvents. It will be understood that in the case of a bulk heterojunction layer the formulation further comprises one or more electron-donating materials.

The formulation may be deposited by any coating or printing method including, without limitation, spin-coating, dip-coating, roll-coating, spray coating, doctor blade coating, wire bar coating, slit coating, ink jet printing, screen printing, gravure printing and flexographic printing.

The one or more solvents of the formulation may optionally comprise or consist of benzene or naphthalene substituted with one or more substituents selected from fluorine, chlorine, C₁₋₁₀ alkyl and C₁₋₁₀ alkoxy wherein two or more substituents may be linked to form a ring which may be unsubstituted or substituted with one or more C₁₋₆ alkyl groups, optionally toluene, xylenes, trimethylbenzenes, tetramethylbenzenes, anisole, indane and its alkyl-substituted derivatives, and tetralin and its alkyl-substituted derivatives.

The formulation may comprise a mixture of two or more solvents, preferably a mixture comprising at least one benzene substituted with one or more substituents as described above and one or more further solvents. The one or more further solvents may be selected from esters, optionally alkyl or aryl esters of alkyl or aryl carboxylic acids, optionally a C₁₋₁₀ alkyl benzoate, benzyl benzoate or dimethoxybenzene. In preferred embodiments, a mixture of trimethylbenzene and benzyl benzoate is used as the solvent. In other preferred embodiments, a mixture of trimethylbenzene and dimethoxybenzene is used as the solvent.

The formulation may comprise further components in addition to the electron-accepting material, the one or more solvents and, in the case of a formulation of forming a bulk heterojunction layer, an electron-donating material. As examples of such components, adhesive agents, defoaming agents, deaerators, viscosity enhancers, diluents, auxiliaries, flow improvers colourants, dyes or pigments, sensitizers, stabilizers, nanoparticles, surface-active compounds, lubricating agents, wetting agents, dispersing agents and inhibitors may be mentioned.

### Applications

A circuit may comprise the OPD connected to one or more of a voltage source for applying a reverse bias to the device; a device configured to measure photocurrent; and an amplifier configured to amplify an output signal of the OPD. The voltage applied to the photodetector may be variable. In some embodiments, the photodetector may be continuously biased when in use.

In some embodiments, a photodetector system comprises a plurality of photodetectors as described herein, such as an image sensor of a camera.

In some embodiments, a sensor may comprise an OPD as described herein and a light source wherein the OPD is configured to receive light emitted from the light source. In some embodiments, the light source has a peak wavelength of at least 900 nm or at least 1000 nm, optionally in the range of 900-1500 nm.

In some embodiments, the light from the light source may or may not be changed before reaching the OPD. For example, the light may be reflected, filtered, down-converted or up-converted before it reaches the OPD.

The organic photoresponsive device as described herein may be an organic photovoltaic device or an organic photodetector. An organic photodetector as described herein may be used in a wide range of applications including, without limitation, detecting the presence and/or brightness of ambient light and in a sensor comprising the organic photodetector and a light source. The photodetector may be configured such that light emitted from the light source is incident on the photodetector and changes in wavelength and/or brightness of the light may be detected, e.g., due to absorption by, reflection by and/or emission of light from an object, e.g. a target material in a sample disposed in a light path between the light source and the organic photodetector. The sample may be a non-biological sample, e.g. a water sample, or a biological sample taken from a human or animal subject. The sensor may be, without limitation, a gas sensor, a biosensor, an X-ray imaging device, an image sensor such as a camera image sensor, a motion sensor (for example for use in security applications) a proximity sensor or a fingerprint sensor. A 1D or 2D photosensor array may comprise a plurality of photodetectors as described herein in an image sensor. The photodetector may be configured to detect light emitted from a target analyte which emits light upon irradiation by the light source or which is bound to a luminescent tag which emits light upon irradiation by the light source. The photodetector may be configured to detect a wavelength of light emitted by the target analyte or a luminescent tag bound thereto.

### Examples

### Synthesis

Example Compound 1 was Synthesised According to Scheme 1

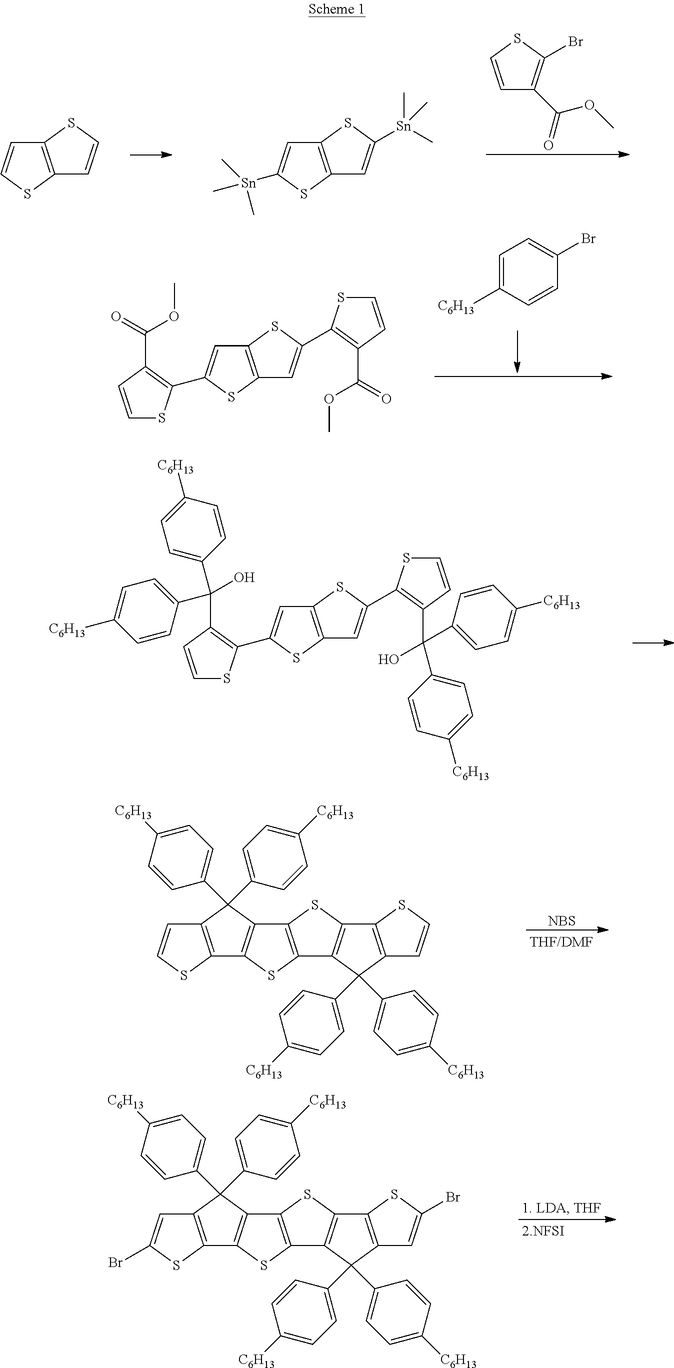

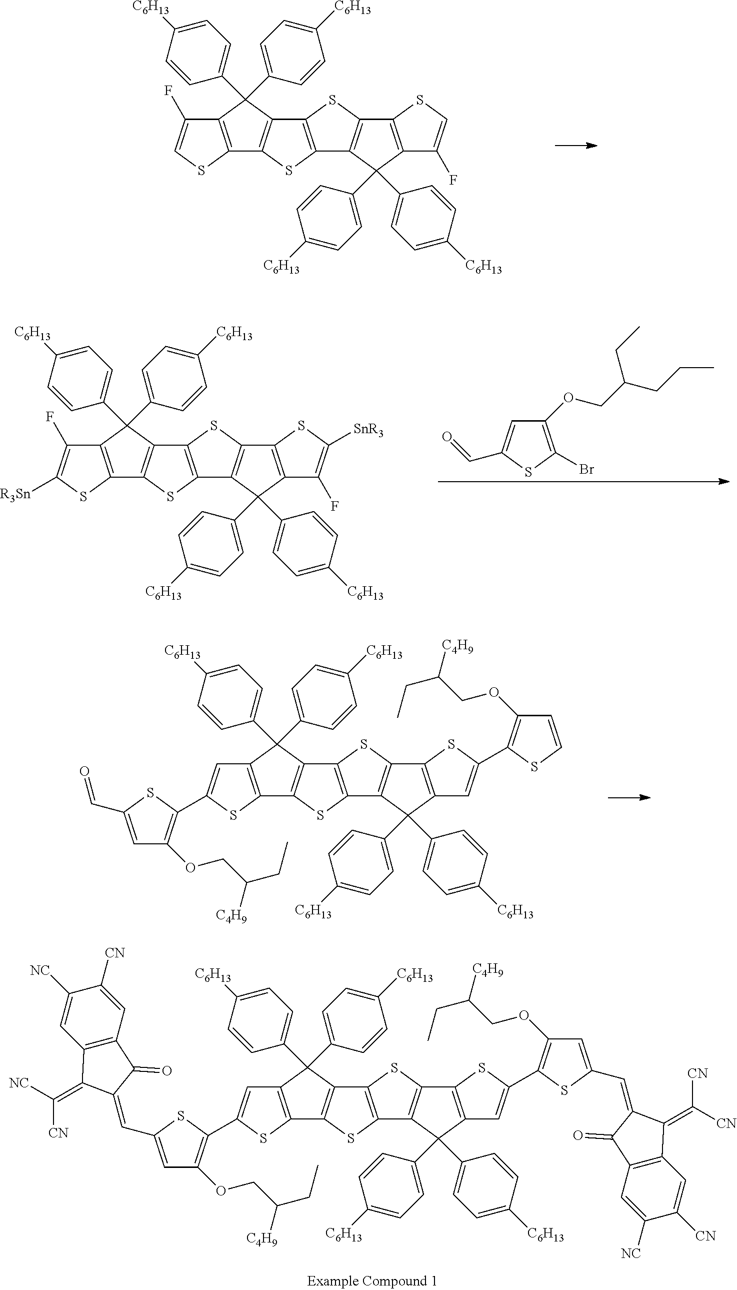

1. A compound of formula (I): 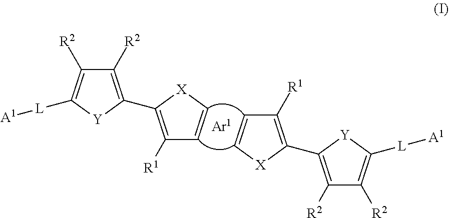
wherein:
Ar¹ is a monocyclic or fused polycyclic group which, together with the rings it is fused to, forms a fused heteroaromatic group;
Y in each occurrence is independently O, S or NR⁵⁵, wherein R⁵⁵ is H or a C₁₋₃₀ hydrocarbyl group;
X in each occurrence is independently O or S;
R¹ in each occurrence is independently H or a substituent with the proviso that at least one R¹ is an electron-withdrawing group;
R² in each occurrence is independently H or a substituent;
L is a direct bond or, together with R², forms an unsubstituted or substituted 5- or 6-membered aromatic or heteroaromatic ring; and
A¹ in each occurrence is an electron-accepting group.

2. The compound according to claim 1, wherein the compound is represented by formula (Ia): 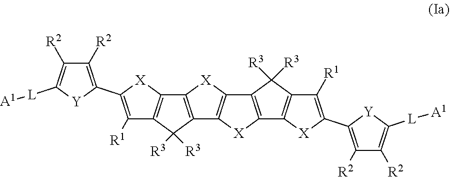
wherein:
Ar¹, Y, X, R¹, R², L, and A¹ are in each occurrence the same as defined in claims 1; and
R³ in each occurrence is independently H or a substituent, optionally wherein each R³ is a substituent.

3. The compound according to claim 1, wherein R¹ in each occurrence is independently H or a substituent selected from the group consisting of alkoxy, aldehydes, ketones, carboxylic acids, esters, halogens, and CN.

4. The compound according to claim 1, wherein R¹ in each occurrence is independently H or a substituent selected from the group consisting of halogens and CN.

5. The compound according to claim 1, wherein R¹ in each occurrence is independently H or a substituent selected from the group consisting of F, Cl and Br.

6. The compound according to claim 1, wherein R² in each occurrence is independently H or a substituent selected from the group consisting of F; CN; NO₂; C₁₋₂₀ alkyl wherein one or more non-adjacent C atoms may be replaced with O, S, NR⁶, COO or CO and one or more H atoms of the alkyl may be replaced with F; phenyl which is unsubstituted or substituted with one or more substituents; and —B(R¹⁴)₂; wherein
R¹⁴ in each occurrence is a substituent, optionally a C₁₋₂₀ hydrocarbyl group; and
R⁶ in each occurrence is independently selected from the group consisting of H; C₁₋₂₀ alkyl wherein one or more non-adjacent C atoms other than the C atom bound to N may be replaced with O, S, NR¹¹, COO or CO and one or more H atoms of the alkyl may be replaced with F;
and phenyl which is unsubstituted or substituted with one or more substituents.

7. The compound according to claim 1, wherein R² in each occurrence is independently H or a substituent selected from the group consisting of C₁₋₂₀ alkyl and C₁₋₁₉ alkoxy.

8. The compound according to claim 1, wherein at least one R² is a substituent;
optionally wherein at least two R²s are a substituent.

9. The compound according to claim 2, wherein when the compound is represented by formula (Ia), R³ in each occurrence is independently H or a substituent selected from the group consisting of C₁₋₂₀ alkyl wherein one or more non-adjacent C atoms may be replaced with O, S, NR⁶, COO or CO and one or more H atoms of the alkyl may be replaced with F; and phenyl which is unsubstituted or substituted with one or more substituents; and wherein
R⁶ in each occurrence is independently selected from the group consisting of H; C₁₋₂₀ alkyl wherein one or more non-adjacent C atoms other than the C atom bound to N may be replaced with O, S, NR¹¹, COO or CO and one or more H atoms of the alkyl may be replaced with F; and phenyl which is unsubstituted or substituted with one or more substituents.

10. The compound according to claim 2, wherein when the compound is represented by formula (Ia), at least one R³ is a substituent; optionally wherein two, three or four R³s are a substituent.

11. The compound according claim 1, wherein at least one A¹ is an electron-accepting group of formula (IXa-2): 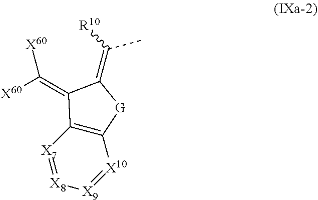
wherein:
G is C═O, C═S SO, SO2, NR³³ or C(R³³)₂ wherein R³³ is CN or COOR⁴⁰ and R⁴⁰ in each occurrence is independently H or a substituent;
each X⁷-X¹⁰ is independently CR¹² or N wherein R¹² in each occurrence is independently H or a substituent selected from C₁₋₂₀ hydrocarbyl and an electron withdrawing group;
R¹⁰ is H or a substituent; and
each X⁶⁰ is independently CN, CF₃ or COOR⁴⁰ wherein R⁴⁰ in each occurrence is independently H or a substituent.

12. The compound according to claim 11, wherein in formula (IXa-2)
G is C═O; and/or
each X⁶⁰ is CN; and/or
each of X⁷-X¹⁰ is CR¹² and each R¹² is independently selected from H or an electron-withdrawing group.

13. The compound according to claim 1, wherein X is S and Y is S.

14. A compound of formula (I): 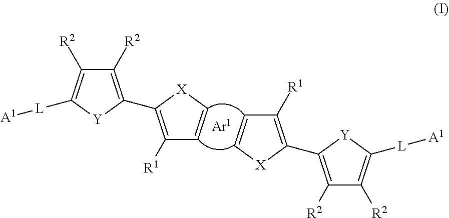
wherein:
Ar¹ is a monocyclic or fused polycyclic group which, together with the rings it is fused to, forms a fused heteroaromatic group;
Y in each occurrence is independently O, S or NR⁵⁵, wherein R⁵⁵ is H or a C₁₋₃₀ hydrocarbyl group;
X in each occurrence is independently O or S;
R¹ in each occurrence is independently H or a substituent;
R² in each occurrence is independently H or a substituent with the proviso that at least one R² is an electron-withdrawing group;
L is a direct bond or, together with R², forms an unsubstituted or substituted 5- or 6-membered aromatic or heteroaromatic ring; and
A¹ in each occurrence is an electron-accepting group.

15. A composition comprising an electron-donating material and an electron-accepting material wherein the electron accepting material is a compound according to claim 1.

16. An organic electronic device comprising an active layer comprising a compound according to claim 1.

17. An organic electronic device according to claim 16, wherein the organic electronic device is an organic photoresponsive device and the active layer is a bulk heterojunction layer disposed between an anode and a cathode of the organic photoresponsive device, and wherein the bulk heterojunction layer comprises a composition comprising an electron-donating material and an electron-accepting material wherein the electron accepting material is a compound of formula (I).

18. An organic electronic device according to claim 17, wherein the organic photoresponsive device is an organic photodetector.

19. A photosensor comprising a light source and an organic photodetector according to claim 18, wherein the organic photodetector is configured to detect light emitted from the light source.

20. A formulation comprising a compound according to claim 1 or a composition comprising an electron-donating material and an electron-accepting material wherein the electron accepting material is a compound of formula (I), dissolved or dispersed in one or more solvents.
